(19) United States
(12) Patent Application Publication
(10) Pub. No.: US 2007/0149487 A1
(43) Pub. Date: Jun. 28, 2007

(54) Antiviral Compositions and Methods

(75) Inventors: Yuan-Ping Pang, Rochester, MN (US); Andrea J. Dooley, Saint Cloud, MN (US); Jewn Giew Park, Rochester, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/557,396

(22) Filed: Nov. 7, 2006

Related U.S. Application Data
(60) Provisional application No. 60/734,600, filed on Nov. 8, 2005.

(51) Int. Cl. A61K 31/655; A61K 31/47; A61K 31/403; A61K 31/381; A61K 31/343

(52) U.S. Cl. 514/150; 514/311; 514/411; 514/443; 514/468

(57) ABSTRACT

Compositions and methods for treating, preventing, or ameliorating one or more symptoms, conditions, or disorders associated with coronavirus infections are provided, in particular small-molecule inhibitors of the chymotrypsin-like cysteine protease of SARS CoV.

### CROSS-REFERENCE TO RELATED APPLICATIONS

This application claims priority under 35 U.S.C. § 119(e) to U.S. Provisional Application No. 60/734,600, filed Nov. 8, 2005. The application is incorporated by reference in its entirety herein.

### STATEMENT AS TO FEDERALLY FUNDED RESEARCH

The work described herein was supported by the National Institute of Allergy and Infectious Diseases (5R01AI054574-02); Defense Advanced Research Projects Agency (DARPA) (DAAD19-01-1-0322); DARPA (DAAD19-03-1-0318); Federal (W81XSH-04-2-0001); and the National Institutes of Health (AI054574). The government may have certain rights in the invention.

### TECHNICAL FIELD

This disclosure relates to small-molecule inhibitors of coronaviruses (CoVs) and Avian Influenza, and more particularly to small-molecule inhibitors of severe acute respiratory syndrome-associated coronavirus (SARS CoV).

### BACKGROUND

Human coronaviruses (CoVs) are responsible for 10% to 30% of all common colds. Most human coronavirus infections result in mild symptoms, and typically fall into one of two groups, 229E or OC43. Severe acute respiratory syndrome (SARS), however, which first appeared in 2002, is an emerging infectious disease with severe mortality (15%). The causative agent of SARS is a previously unrecognized human coronavirus called SARS-associated coronavirus (SARS-CoV). The SARS-CoV genome encodes a chymotrypsin-like cysteine proteinase (CCP, also known as M^{pro} or 3CL^{pro}) that proteolytically processes polypeptides required for viral replication and transcription, thus representing a drug target for treating SARS. Although small-molecule inhibitors of CCP have been identified, the development of these inhibitors as clinical drugs for treating SARS has not yet been achieved. Other mammalian or avian CoVs can also cause moderate to severe infections in both domesticated and wild animals.

### SUMMARY

This disclosure provides materials and methods for treating, preventing, or ameliorating one or more symptoms, disorders, or conditions associated with coronavirus (CoV) infections, including mammalian CoV infections (e.g., human OC43 CoV, human 229E CoV, human SARS CoV, Himalayan palm civet coronavirus (SARS CoV Strain SZ16), canine coronavirus (CCoV), porcine epidemic diarrhea virus (PEDV), transmissible gastroenteritis virus (TGEV), bovine coronavirus (BCoV), equine coronavirus (ECoV), murine hepatitis virus (MHV), porcine haemmagglutinating encephalomyelitis virus (PHEV), puffinosis virus, rat sialodacryoadenitis coronavirus (SDAV), and feline infectious peritonitis virus (FIPV); avian CoV infections (e.g., infectious bronchitis virus (IBV) and turkey coronavirus (TCoV.

In some embodiments, small-molecule inhibitors of human SARS CoV are provided. A small-molecule inhibitor can inhibit the chymotrypsin-like cysteine protease (CCP) of a CoV, such as the CCP of SARS CoV Methods for using such small-molecule inhibitors to treat, prevent, or ameliorate one or more symptoms of CoVs or disorders associated with CoVs, including sore throat, congestion, pneumonia, severe respiratory distress, upper or lower respiratory infection, coughing, sneezing, runny nose, fever, body aches, and shortness of breath are also provided. Kits and articles of manufacture containing one or more small-molecule inhibitors and accessory items are also provided.

This disclosure also provides materials and methods for treating, preventing, or ameliorating one or more symptoms, disorders, or conditions associated with Highly Pathogenic Avian Influenza (HPAI, or Bird Flu) infections, e.g., influenza A viruses including subtypes H5, H7, and in particular H5N1. Pharmacophore analyses using the current Avian Influenza drug, Tamiflu™, suggest that the presently-described small-molecules may be useful for inhibiting Avian Influenza infections.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 provides the chemical structures and protonation states of small-molecule SARS CCP inhibitor compounds identified using a 4-ns molecular dynamics simulation model.

FIG. 2 is an overlay of CS11 (yellow) and Tamiflu™ (green) demonstrating their structural similarity.

FIG. 3 sets forth various embodiments of Formulas I(a) and I(b), as described herein.

FIG. 4 sets forth various embodiments of Formulas II(a) and II(b), as described herein.

FIG. 5 sets forth various embodiments of Formulas III and IV, as described herein.

FIG. 6 provides synthetic schemes for the preparation of compounds of Formulas I-IV.

FIG. 7 shows the chemical structure of Tamiflu™ (oseltamivir free base); Tamiflu™ is also prepared as the phosphate salt, oseltamivir phosphate, having the chemical name (3R, 4R, 5S)-4-acetylamino-5-amino-3-(1-ethylpropoxy)-1-cyclohexene-1-carboxylic acid, ethyl ester, phosphate (1:1).

### DETAILED DESCRIPTION

Provided herein are small-molecule inhibitors of CoVs, including mammalian and avian CoVs. The small-molecule inhibitors were designed to inhibit the chymotrypsin-like cysteine protease (CCP) of SARS CoV using, in part, information obtained from molecular modeling studies of the protease and its active site. Based on pharmacophore analysis, the compositions may also be useful for inhibiting Avian Influenza infections.

A. Definitions

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a CoV infection, e.g., SARS CoV, are ameliorated or otherwise beneficially altered. Treatment also encompasses any pharmaceutical use of the compositions herein, such as uses for treating diseases, disorders, or ailments in which a CoV is implicated. In addition, treatment can mean any manner in which one or more of the symptoms of an Avian Influenza infection are ameliorated or otherwise beneficially altered.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response in an assay that measures such response.

As used herein, the term Kᵢ represents the dissociation constant of an enzyme/inhibitor complex. It is theoretically independent of the substrate against which the inhibitor is tested. Kᵢ can be calculated from an IC₅₀ using the equation: Kᵢ=IC₅₀*Kₘ/(S+Kₘ), where S is the concentration of substrate, and Kₘ is the substrate concentration (in the absence of inhibitor) at which the velocity of the reaction is half-maximal. The Kᵢ of an inhibitor for inhibition of a particular substrate (fixed Kₘ) is constant.

As used herein, EC₅₀ refers to a drug concentration that produces 50% of inhibition, and CC₅₀ refers to a drug concentration that produces 50% of toxicity.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. In certain cases, particular R and S configurations may be preferred, e.g., the configurations at positions 2, 3 and 4 of the six-membered heterocycle of Formula III can be R, S and S, respectively. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” refer to carbon chains that may be straight or branched. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl).

As used herein, “cycloalkyl” refers to a saturated mono- or multi-cyclic ring system, in certain embodiments of 3 to 10 carbon atoms, in other embodiments of 3 to 6 carbon atoms. The ring systems of the cycloalkyl groups may be composed of one ring or two or more rings which may be joined together in a fused, bridged or spiro-connected fashion. Examples include, but are not limited to, cyclopropyl, cyclobutyl, cyclopentyl, and cyclohexyl.

As used herein, “cyclohexenyl” refers to a cyclohexyl ring with one double bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members, where one or more, in one embodiment 1 to 4, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an alkyl group in which one or more of the hydrogen atoms are replaced by halogen.

As used herein, “carboxy” refers to a divalent radical, —C(O)O—.

As used herein, “aminocarbonyl” refers to —C(O)NH₂.

As used herein, “aminoalkyl” refers to —RNH₂, in which R is alkyl.

As used herein, “alkoxy” and “alkylthio” refer to RO— and RS—, in which R is alkyl.

As used herein, “aryloxy” and “arylthio” refer to RO— and RS—, in which R is aryl.

As used herein, “amido” refers to the divalent group —C(O)NH—.

As used herein, “hydrazide” refers to the divalent group —C(O)NHNH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Compounds

The compounds provided herein exhibit cell-based ex vivo activity against SARS CoV and associated disorders. For example, the compounds provided herein can rescue cells from infection of SARS CoV. In one embodiment, the compounds treat, prevent, or ameliorate one or more symptoms associated with CoV infection, including SARS CoV infection. In certain embodiments, the compounds inhibit the protease activity of a CCP from a CoV, including the SARS CoV CCP.

Use of any of the compounds provided herein, or their pharmaceutically acceptable salts or derivatives, in the treatment, prevention, or amelioration of CoV infections (e.g., SARS CoV infection) or Avian Influenza infections, is also provided, as well as use of any of the compounds, or pharmaceutically acceptable salts or derivatives thereof, in the preparation of a medicament for the treatment, prevention, or amelioration of a CoV infection, such as SARS CoV infection, or an Avian Influenza infection.

Compounds for use in the compositions and methods provided herein can have Formula I(a) or I(b) below.

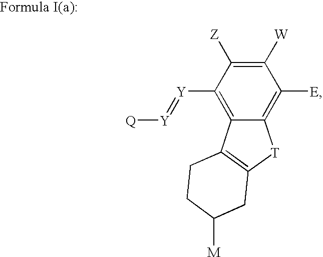

or a pharmaceutically acceptable salt or derivative thereof,

wherein Q is selected from:

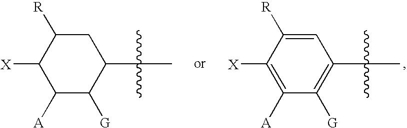

Y is N or CH;

X=H, CO₂H, NO₂, CH₂OH, COCH₃, CONH₂, or CONHCH₃;

Z=H, OH, F, NH₂, or aminoalkyl having from 1 to 5 carbon atoms;

A=H, or alkyl having from 1 to 5 carbon atoms;

G=H, or alkyl having from 1 to 5 carbon atoms;

R=H, OH, F, O(CH₂)ₘN(CH₃)₂, O(CH₂)ₘN(CH₃)H, O(CH₂)ₘNH₂, wherein m=1 to 5;

T is O, S, NH, or CO, or T is not present, in which case the adjacent cyclohexenyl ring is a cyclohexyl ring;

M=H or alkyl having from 1 to 3 carbon atoms, provided that if M=H, W=H, E=H, and T=O, then Z is not OH;

W=H, (CH₂)ₘNH₂, or (CH₂)ₘCONH₂, wherein m=1 to 5; and

E=H, alkyl having from 1 to 3 carbon atoms, CH₂CONH₂, or CH₂CONHR′, wherein R′ is alkyl having from 1 to 4 carbon atoms; or W and E together form a cyclohexyl ring which is fused to the adjacent phenyl ring, wherein said cyclohexyl ring is optionally substituted with X.

In some embodiments of Formula I(a), one Y is N and one Y is CH. In some embodiments, T is O or S. In some embodiments, X is CO₂H. In some embodiments, M is methyl. In some embodiments, W and E form a cyclohexyl ring substituted with X. In some embodiments, G is methyl. In some embodiments, G is ethyl. In some embodiments, T is not present and the cyclohexenyl group is a cyclohexyl group. In some embodiments, Z is OH. In some embodiments, Z is F. In some embodiments, Z is NH₂. In some embodiments, R is F. In some embodiments, R is OH. In some embodiments, A is methyl. In some embodiments, A is ethyl.

Compounds of Formula I(b) are also provided:

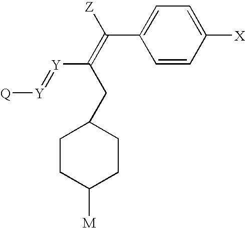

or a pharmaceutically acceptable salt or derivative thereof,

wherein Q is selected from:

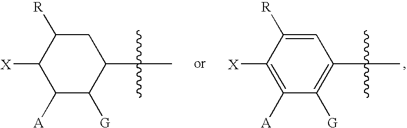

Y is N or CH;

X=H, CO₂H, NO₂, CH₂OH, COCH₃, CONH2, or CONHCH₃;

Z=H, OH, F, NH₂, or aminoalkyl having from 1 to 5 carbon atoms;

A=H, or alkyl having from 1 to 5 carbon atoms;

G=H, or alkyl having from 1 to 5 carbon atoms;

R=H, OH, F, O(CH₂)ₘN(CH₃)₂, O(CH₂)ₘN(CH₃)H, O(CH₂)ₘNH₂, wherein m=1 to 5; and

M=H or alkyl having from 1 to 3 carbon atoms.

In some embodiments of Formula I(b), one Y is CH and one Y is N. In some embodiments, X is CO₂H. In some embodiments, M is methyl. In some embodiments, G is methyl. In some embodiments, G is ethyl. In some embodiments, Z is OH. In some embodiments, Z is F. In some embodiments, Z is NH₂.

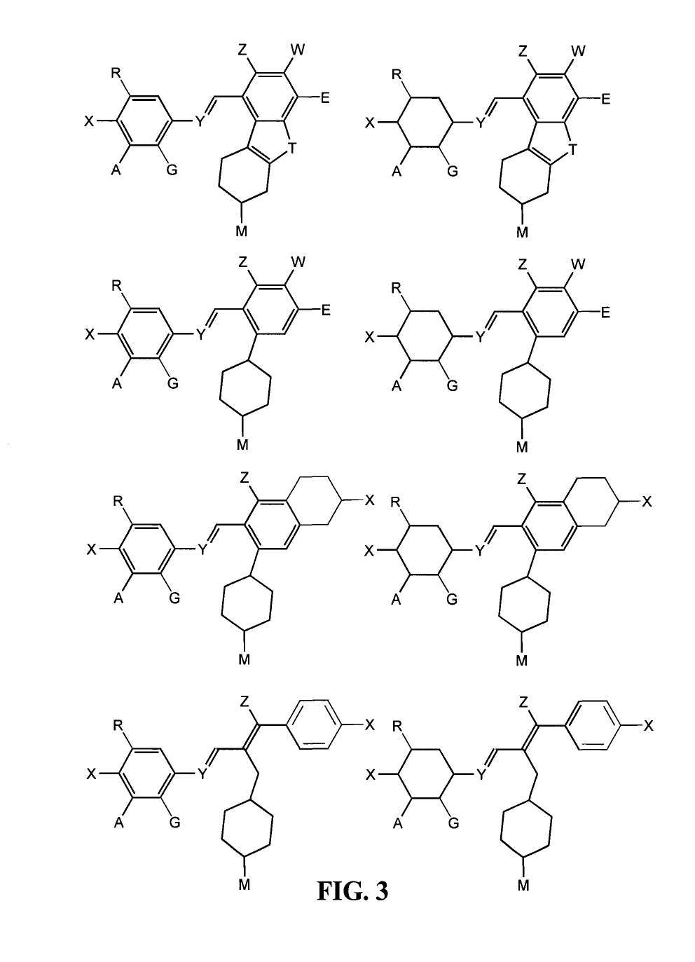

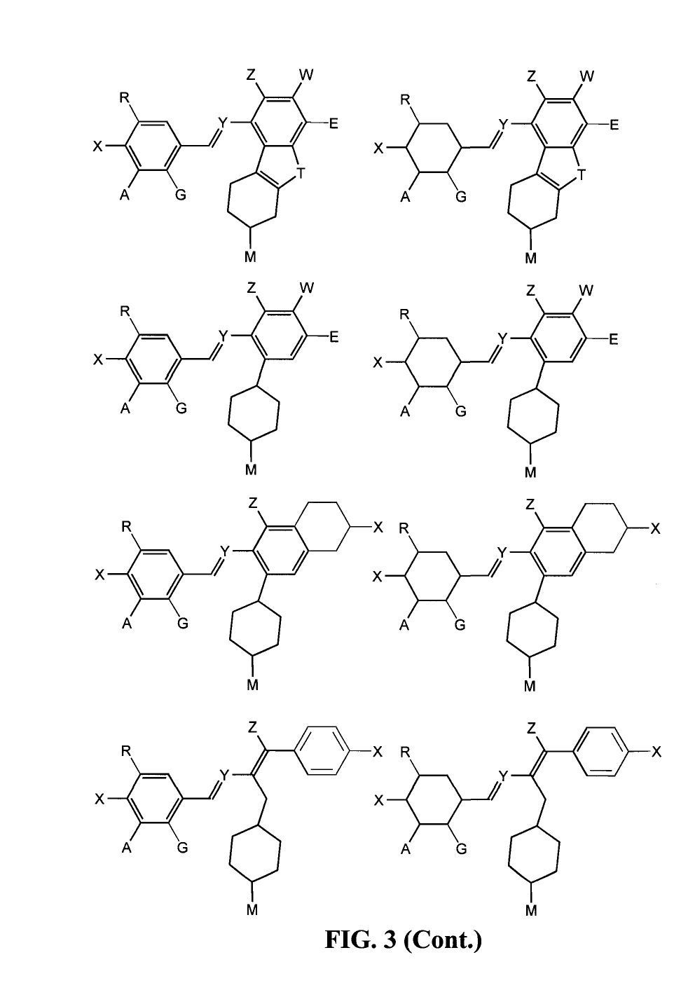

Compounds of Formula I(a) or I(b) having the formulae as set forth in FIG. 3 are also provided, wherein the substituents are as provided above.

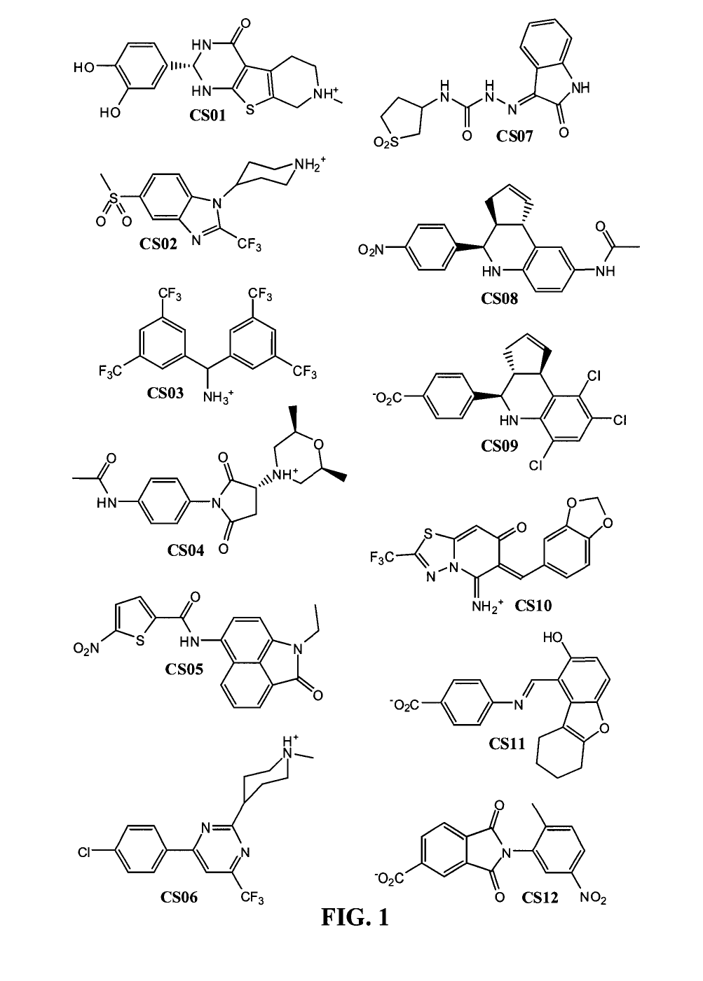

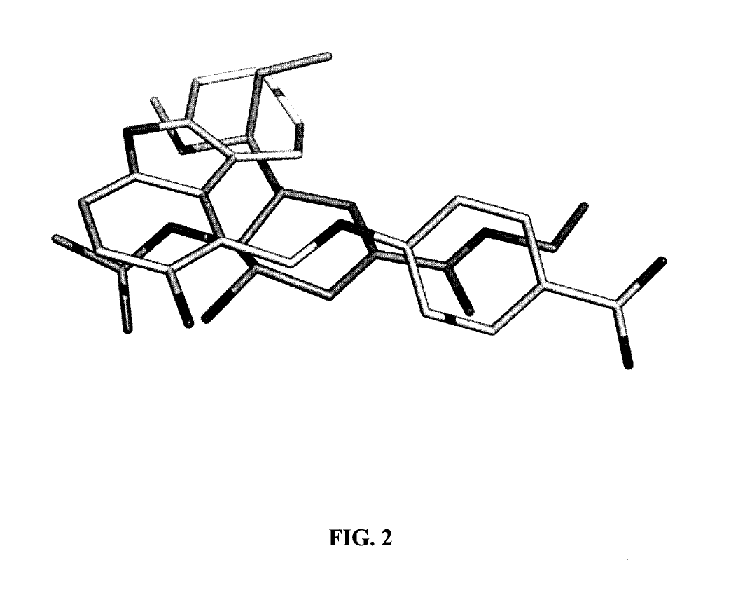

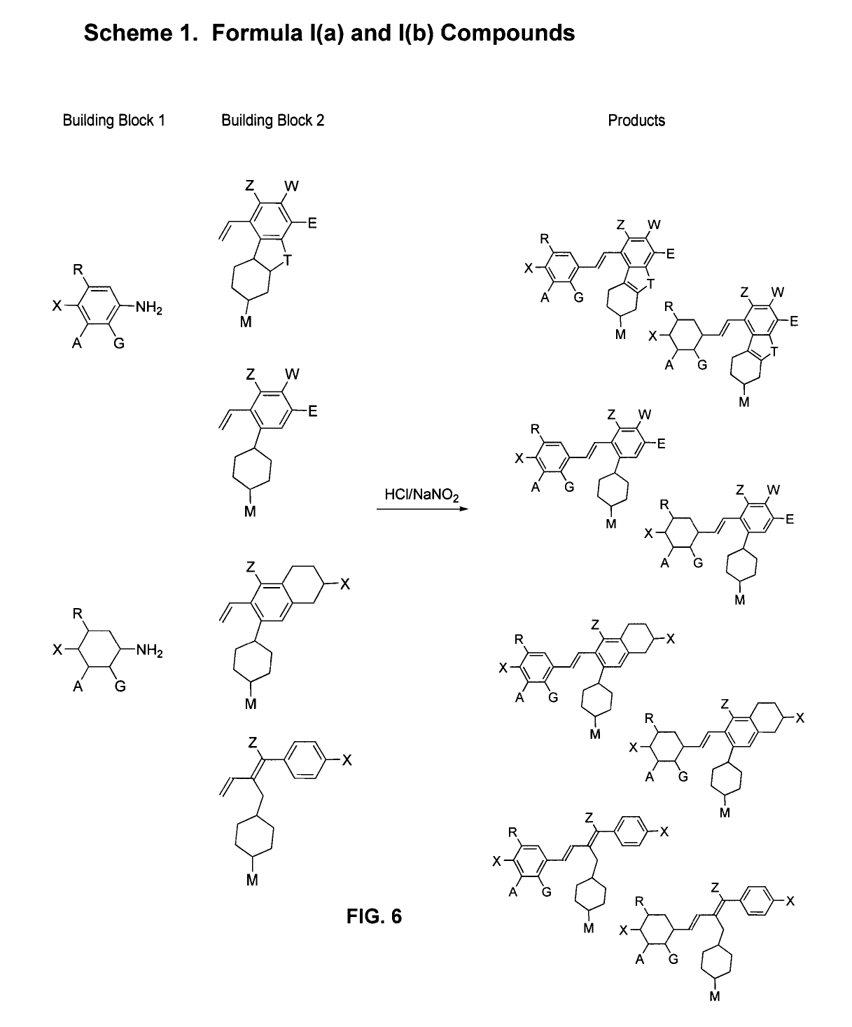

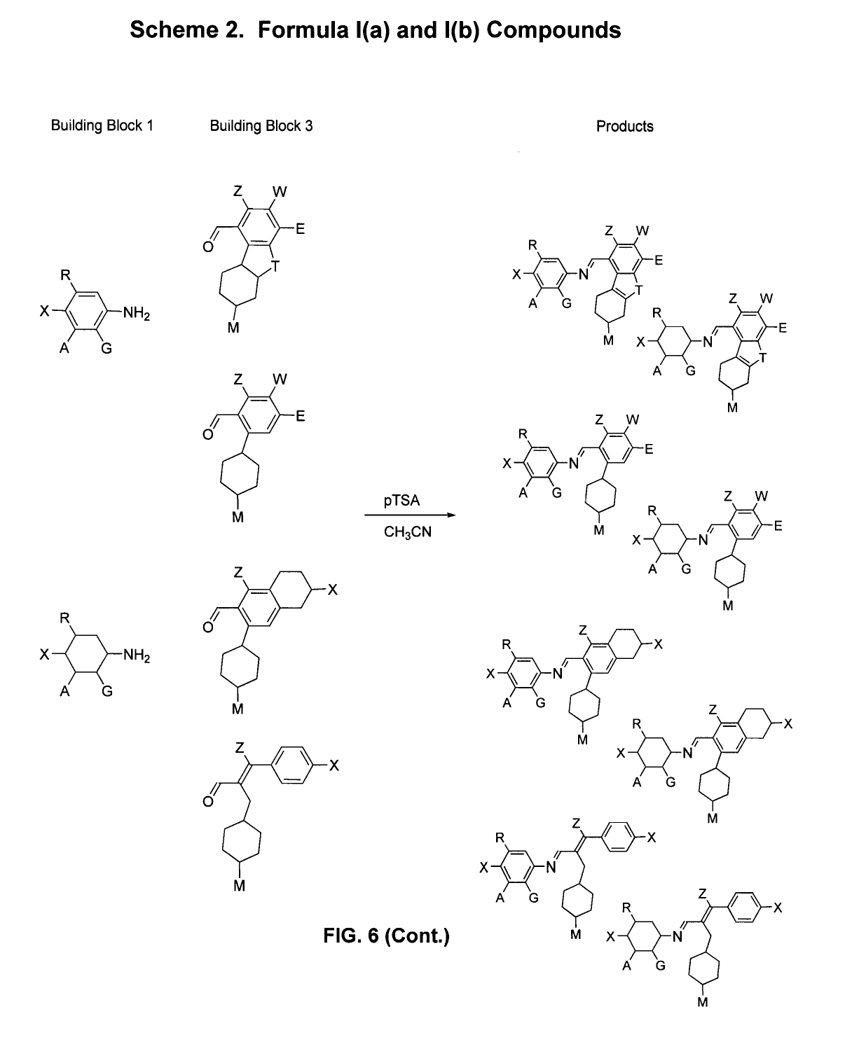

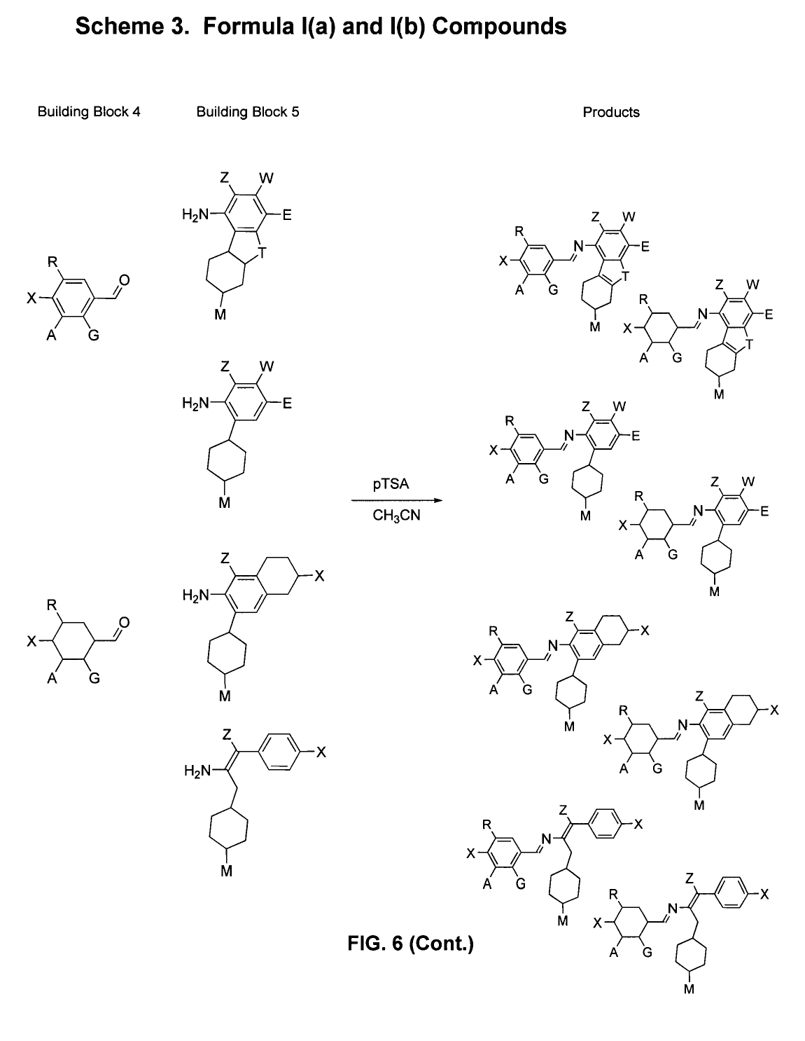

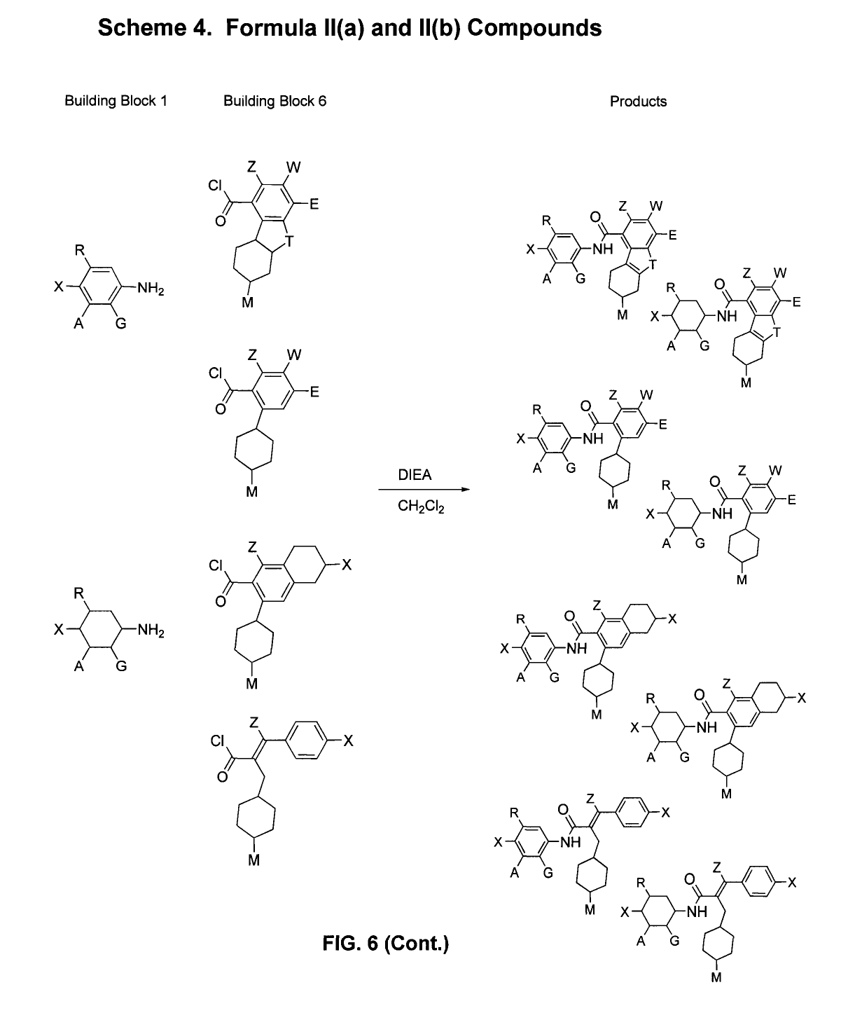

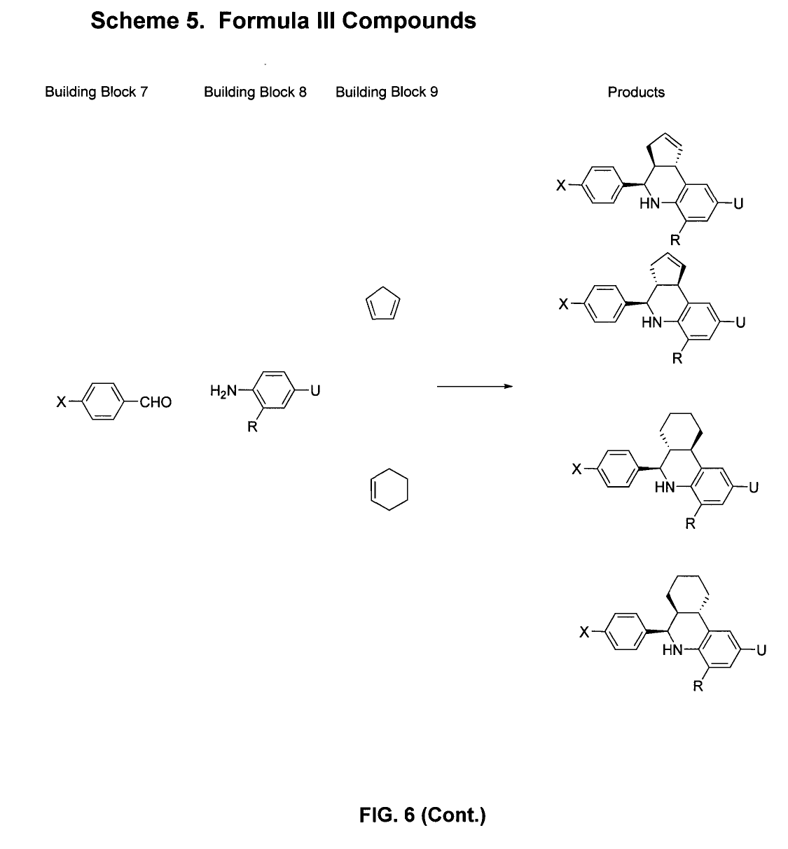

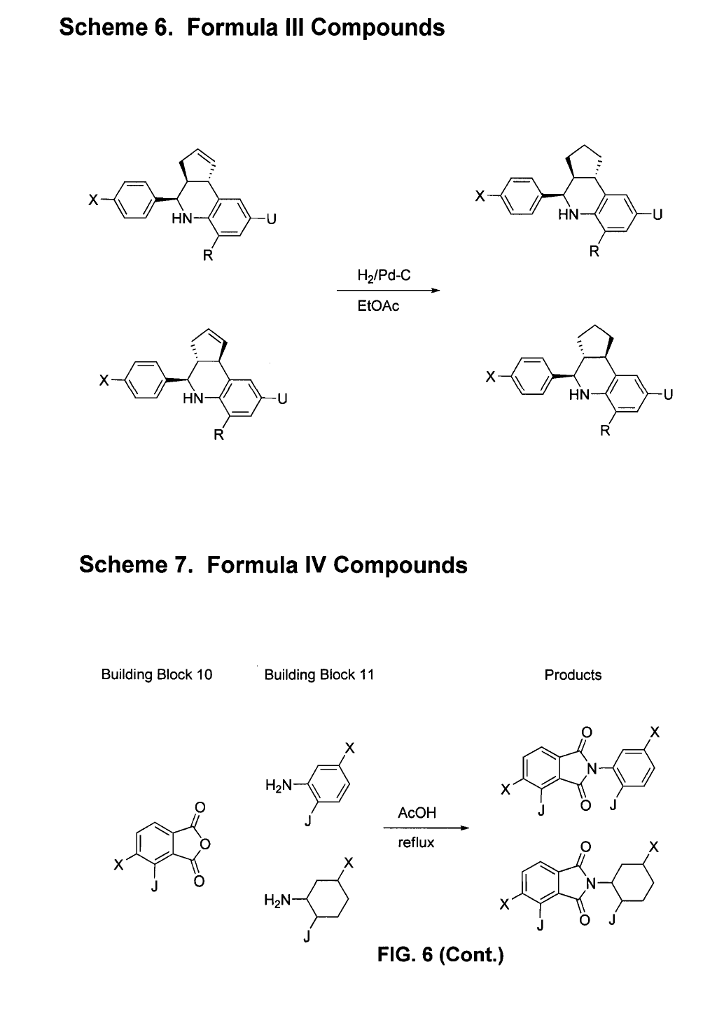

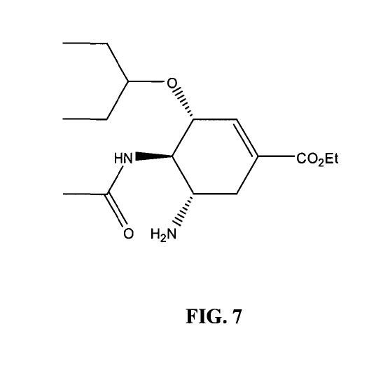

In other embodiments, compound CS11 as set forth in FIG. 1 can be used in some of the compositions and methods described herein. Methods for preparing compounds of Formula I(a) or I(b) are set forth in FIG. 6.

Compounds for use in the compositions and methods provided herein can have Formula II(a) or II(b) below.

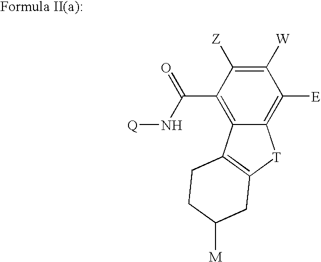

or a pharmaceutically acceptable salt or derivative thereof,

wherein Q is selected from:

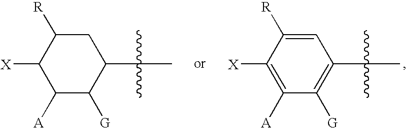

X=H, CO₂H, NO₂, CH₂OH, COCH₃, CONH2, or CONHCH₃;

Z=H, OH, F, NH₂, or aminoalkyl having from 1 to 5 carbon atoms;

A=H, or alkyl having from 1 to 5 carbon atoms;

G=H, or alkyl having from 1 to 5 carbon atoms;

R=H, OH, F, O(CH₂)ₘN(CH₃)₂, O(CH₂)ₘN(CH₃)H, O(CH₂)ₘNH₂, wherein m=1 to 5;

T is O, S, NH, or CO, or T is not present, in which case the adjacent cyclohexenyl ring is a cyclohexyl ring;

M=H or alkyl having from 1 to 3 carbon atoms;

W=H, (CH₂)ₘNH₂, or (CH₂)ₘCONH₂, wherein m=1 to 5; and

E=H, alkyl having from 1 to 3 carbon atoms, CH₂CONH₂, or CH₂CONHR′, wherein R′ is alkyl having from 1 to 4 carbon atoms; or W and E together form a cyclohexyl or phenyl ring which is fused to the adjacent phenyl ring, wherein said cyclohexyl or phenyl ring is optionally substituted with X.

In some embodiments of Formula II(a), T is O or S. In some embodiments, X is CO₂H. In some embodiments, M is methyl. In some embodiments, W and E form a cyclohexyl ring substituted with X. In some embodiments, G is methyl. In some embodiments, G is ethyl. In some embodiments, T is not present and the cyclohexenyl group is a cyclohexyl group. In some embodiments, Z is OH. In some embodiments, Z is F. In some embodiments, Z is NH₂. In some embodiments, R is F. In some embodiments, R is OH. In some embodiments, A is methyl. In some embodiments, A is ethyl.

Formula II(b) can have the following structure:

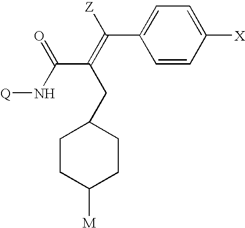

wherein Q is selected from:

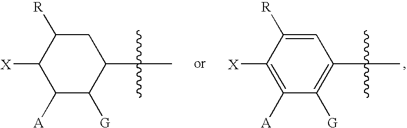

wherein X=H, CO₂H, NO₂, CH₂OH, COCH₃, CONH₂, or CONHCH₃;

Z=H, OH, F, NH₂, or aminoalkyl having from 1 to 5 carbon atoms;

A=H, or alkyl having from 1 to 5 carbon atoms;

G=H, or alkyl having from 1 to 5 carbon atoms;

R=H, OH, F, O(CH₂)ₘN(CH₃)₂, O(CH₂)ₘN(CH₃)H, O(CH₂)ₘNH₂, wherein m=1 to 5; and

M=H or alkyl having from 1 to 3 carbon atoms.

In some embodiments of Formula II(b), X is CO₂H. In some embodiments, M is methyl. In some embodiments, G is methyl. In some embodiments, G is ethyl. In some embodiments, Z is OH. In some embodiments, Z is F. In some embodiments, Z is NH₂. In some embodiments, A is methyl. In some embodiments, A is ethyl.

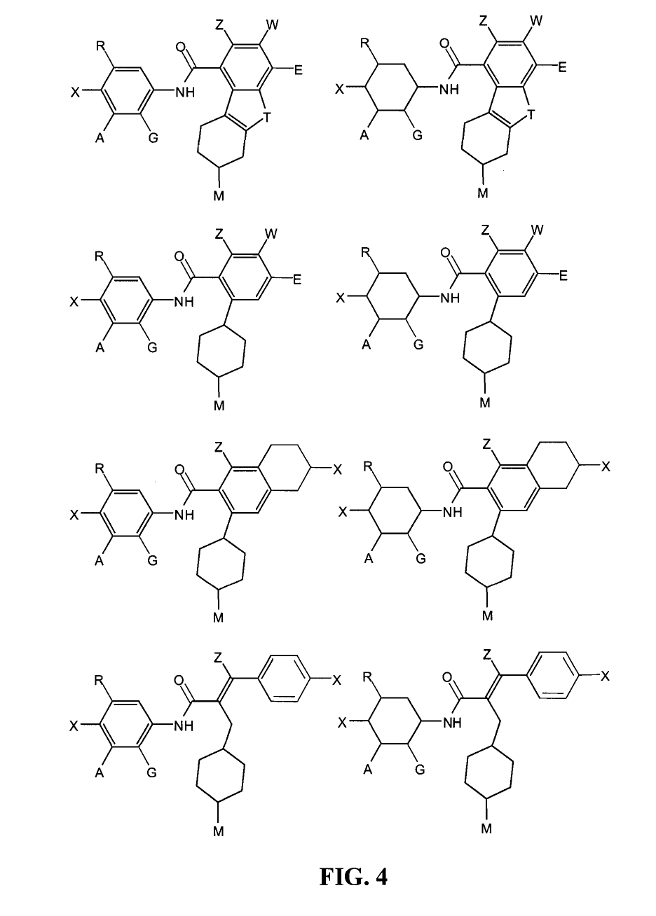

Compounds of Formula II(a) or II(b) having the formulae as set forth in FIG. 4 are also provided herein, wherein the substituents are as provided previously. Methods for preparing compounds of Formula II(a) or II(b) are set forth in FIG. 6.

Compounds for use in the compositions and methods provided herein can have Formula III, below:

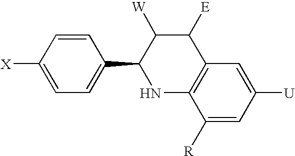

or a pharmaceutically acceptable salt or derivative thereof, wherein:

W and E together form a 5- or 6-membered cycloalkyl ring that is saturated or unsaturated and that is fused in any stereochemistry relative to the adjacent heterocyclyl ring;

X=H, CO₂H, NO₂, CH₂OH, COCH₃, CONH2, or CONHCH₃;

R=H, OH, F, O(CH₂)ₘN(CH₃)₂, O(CH₂)ₘN(CH₃)H, O(CH₂)ₘNH₂, wherein m=1 to 5; and

U=H, F, NH₂, NHR′, NHC(═O)R′, or N(R′)C(═O)R′, wherein R′ is alkyl having from 1 to 4 carbon atoms, provided that if U is NHC(═O)R′, where R′ is methyl, and R is H, then X is not NO₂.

In some embodiments, W and E form a 5-membered cycloalkyl ring. In some embodiments, W and E form a 6-membered cycloalkyl ring. In some embodiments, W and E form a 5-membered cycloalkyl ring that is unsaturated, e.g., having one double bond. In some embodiments, W and E form a 6-membered cycloalkyl ring that is unsaturated, e.g., having one double bond. In some embodiments, X is CO₂H. In some embodiments, X is NO₂. In some embodiments, U is F. In some embodiments, U is NH₂. In some embodiments, U is NHC(═O)C₂H₅. In some embodiments, R is OH. In some embodiments, Formula III has the structure:

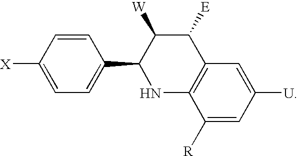

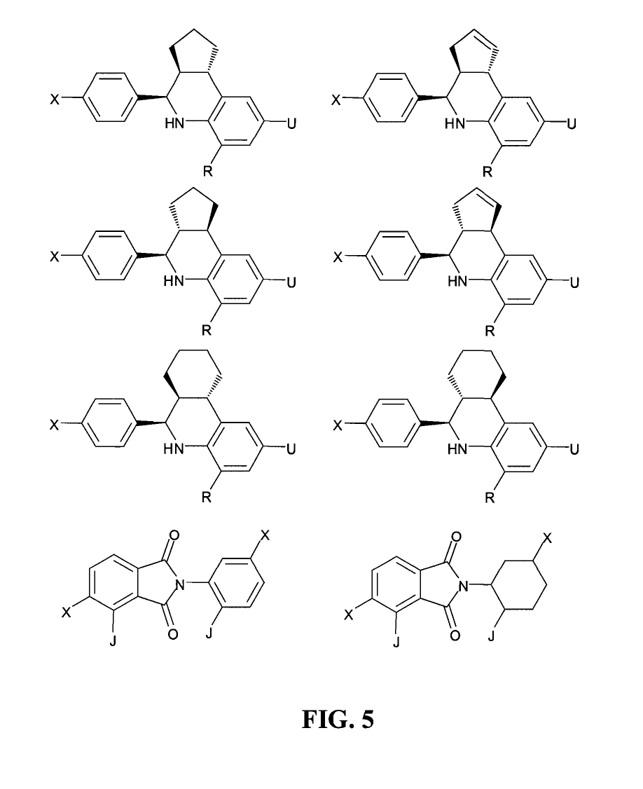

Compounds of Formula III having the formulae as set forth in FIG. 5 are also provided herein, wherein the substituents are as provided previously. For example, various stereochemistries of the rings formed by W and E are depicted in FIG. 5.

In other embodiments, compounds CS08 and CS09 as set forth in FIG. 1 can be used in the compositions and methods described herein. Methods for preparing compounds of Formula III are set forth in FIG. 6.

Compounds for use in the compositions and methods provided herein can have Formula IV, below:

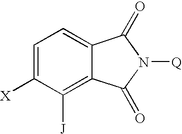

or a pharmaceutically acceptable salt or derivative thereof,

wherein Q is:

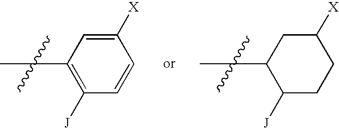

wherein X=H, CO₂H, CH₂OH, COCH₃, CONH2, or CONHCH₃; and

J=H or alkyl having from 1 to 5 C atoms.

In some embodiments, J is methyl. In some embodiments, J is ethyl. In some embodiments, J is iso-propyl. In some embodiments, X is CO₂H.

Compounds of Formula IV having the formulae as set forth in FIG. 5 are also provided herein, wherein the substituents are as provided previously.

In other embodiments, compound CS12 as set forth in FIG. 1 can be used in the compositions and methods described herein. Methods for preparing compounds of Formula IV are provided in FIG. 6.

C. Preparation of the Compounds

The compounds for use in the compositions and methods provided herein may be obtained from commercial sources (e.g., Asinex, Array BioPharma, Bionet, ChemBridge, ChemDiv, Enamine, Interbioscreen, Microchem Ltd, Maybridge, Peakdale, Sigma-Aldrich, Specs Biospecs, and TimTec) or may be prepared by methods well known to those of skill in the art or by the methods shown herein (e.g., see FIG. 6). One of skill in the art would be able to prepare all of the compounds for use herein by routine modification of these methods using the appropriate starting materials.

D. Formulation of Pharmaceutical Compositions

The pharmaceutical compositions provided herein contain therapeutically effective amounts of one or more of the compounds provided herein that are useful in the treatment, prevention, or amelioration of one or more of the symptoms associated with CoV infection (e.g., SARS CoV infection), or a disorder, condition, or ailment in which CoV infection (e.g., SARS CoV infection) is implicated, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. For example, the compounds may be formulated or combined with known antiviral compounds (e.g., Tamiflu™, TF2B, TF3, VIRA38, Viracept, and/or Agenerase), NSAIDs, anti-inflammatory compounds, steroids, and/or antibiotics.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of CoV infection, e.g., SARS CoV infection.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro, ex vivo and in vivo systems, and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disorder being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof, and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action. The active ingredient is a compound or pharmaceutically acceptable derivative thereof as described herein. Higher concentrations, up to about 98% by weight of the active ingredient, may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

2. Injectables, Solutions, and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration should be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010,715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture (e.g., kits) containing packaging material, a compound or pharmaceutically acceptable derivative thereof provided herein within the packaging material, and a label that indicates that the compound or composition, or pharmaceutically acceptable derivative thereof, is useful for treatment, prevention, or amelioration of one or more symptoms or disorders in which CoV infection, including SARS infection, is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

8. Sustained Release Formulations

Also provided are sustained release formulations to deliver the compounds to the desired target at high circulating levels (between 10⁻⁹ and 10⁻⁴ M). The levels are either circulating in the patient systemically, or in one embodiment, localized to a site of, e.g., paralysis.

It is understood that the compound levels are maintained over a certain period of time as is desired and can be easily determined by one skilled in the art. Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

E. Evaluation of the Activity of the Compounds

The activity of the compounds provided herein as inhibitors of CoV infection (e.g., SARS CoV infection), or CCP protease activity, may be measured in standard assays, e.g., X-ray crystallographic analysis of inhibitor-bound CCP complexes, enzymatic inhibition assays, and cell cytoprotection and viability assays (as described below).

F. Methods of Use of the Compounds and Compositions

Provided herein are methods to treat, prevent, or ameliorate symptoms, conditions, or disorders associated with mammalian and avian CoV infections, including human OC43 and 229E CoV infections; human SARS CoV infection, FIPV infection, CCoV infection, and others as identified above. Also provided are methods to inhibit CoV protease activity, including CCP protease activity, such as SARS CCP activity. In certain cases, the methods can be used to counter-effect CoV or Avian Influenza infection from biological-based weapons.

The methods include administering one or more of the compounds described herein, or a pharmaceutically acceptable salt or derivative thereof, to a mammal, e.g., a human, cat, dog, horse, pig, cow, sheep, mouse, rat, or monkey; to a bird, e.g., a goose, duck, chicken, turkey, pheasant, grouse, swan, pigeon, crow, raven, eagle, and gull. Any of the compounds described herein can be administered to a mammal or a bird for use in a method, e.g., compounds according to Formula I(a), I(b), II(a), II(b), III, or IV, or compounds CS08, CS09, CS11, CS12, or CS14. Administration of combinations of such compounds are also contemplated, as well as combinations with other compounds, as described further herein.

In certain embodiments, the symptoms, conditions, or disorders associated with CoV infection include one or more of the following: difficulty breathing, pneumonia, secondary pneumonia, severe respiratory distress, nasal congestion, coughing, lung congestion, asphyxiation, suffocation, fatigue, dizziness, fever, chills, body aches, sore throat, nausea, vomiting, abdominal pain, and diarrhea.

In practicing the methods, effective amounts of the compounds or compositions provided herein are administered. Such amounts are sufficient to achieve a therapeutically effective concentration of the compound or active component of the composition in vivo.

Any of the compounds or compositions containing one or more compounds provided herein can be administered before, simultaneously with, or after administration of other drugs, such as antiviral drugs (e.g., Tamiflu™, TF2B, TF3, VIRA38, Viracept, and/or Agenerase), NSAIDs, anti-inflammatory drugs, steroids, and/or antibiotics.

### EXAMPLES

Materials and Methods

Models of CCP. The 2.0/4.0-ns computer model was obtained by performing 200 molecular dynamics simulations (2.0/4.0 ns for each simulation with a 1.0-fs time step and different initial velocities) of a reported substrate fragment-bound CCP model (Pang, Y.-P., Three-dimensional model of a substrate-bound SARS chymotrypsin-like cysteine proteinase predicted by multiple molecular dynamics simulations: catalytic efficiency regulated by substrate binding. Proteins. 57, 747-757 (2004)), followed by averaging 100,000 instantaneous structures of the CCP model derived at 1.0 ps intervals during the last 500-ps period of the 200 simulations. The average structures for the 4.0-ns and 2.0-ns computer models were obtained with and without a root-mean-square fit of the backbones of residues 1-308 of the 100,000 instantaneous structures, respectively. The two crystal structures used in the virtual screens were modified from the available crystal structures at the Protein Data Bank (PDB codes: 1UK4 and 1UK2) (Yang, H. T. et al., The crystal structures of severe acute respiratory syndrome virus main protease and its complex with an inhibitor. Proc. Natl. Acad. Sci. U.S.A. 100, 13190-13195 (2003)) by adding hydrogen atoms and energy minimizing the hydrogen atoms with a positional constraint applied to all non-hydrogen atoms of CCP.

Virtual Screening. Two-stage docking of 361,413 relatively rigid, unique, small molecules into the active site of CCP was carried out by the EUDOC program (Pang, Y. P., Perola, E., Xu, K. & Prendergast, F. G., EUDOC: A computer program for identification of drug interaction sites in macromolecules and drug leads from chemical databases, J. Comp. Chem. 22, 1750-1771 (2001)), performed on a dedicated cluster of 800 Intel Xeon P4 processors (2.2/2.4 Ghz) according to the published protocol (Perola, E. et al. Successful virtual screening of a chemical database for farnesyltransferase inhibitor leads. J. Med. Chem. 43, 401-408 (2000)). The translational and rotational increments at the first stage were 1.0 Å and 10 degrees of arc, respectively, and default increments were used at the second stage. The relatively rigid molecules were selected from an in-house database of 2.5 million small molecules using the criterion that each selected molecule has no more than four conformation-governing torsions. All small molecules to be screened were protonated or deprotonated according to physiological pH of 7.4 and their three-dimensional structures and atomic charges were obtained from AM1 semi-empirical calculations. Conformations of CCP and small molecules were not allowed to change during docking. A docking box (3.0×10.0×2.5 Å³) that confines the translation of the mass centre of each molecule within the active site of CCP was defined in the area surrounded by residues 22-28, 38-43, 46-51, 140-148, 163-169, and 183-194.

Chemicals. All tested compounds were purchased from Asinex, Array BioPharma, Bionet, ChemBridge, ChemDiv, Enamine, Interbioscreen, Microchem Ltd, Maybridge, Peakdale, Sigma-Aldrich, Specs Biospecs, and TimTec, and were confirmed by NMR spectroscopic analysis. Mass spectrometry analysis of chemical structures was performed when necessary.

Cell-Based Assays. Vero E6 cells were prepared in Dulbecco's modified Eagle medium without Phenol red, and supplemented with 5% FBS, 2% L-glutamine, and 1% P/S. Cells were seeded at 1×10⁴ cells/well density in 96-well, black clear bottom plates. Cells were allowed to adhere to the plates overnight in an incubator with 5% CO₂ at 37° C. Stock solutions of computer-identified compounds through the 4.0-ns and 2.0-ns models were prepared at 20 and 60 mM in 100% DMSO, respectively. Compounds identified with the 4.0-ns model were tested at concentrations of 100 μM and 6 downward 1/2-log dilutions. Compounds identified with the 2.0-ns model were tested initially at 100 μM, and then at concentrations of 300 μM and 6 downward 1/2-log dilutions for those which showed activity at 100 μM. Calpain was used as a positive control with the highest dose at 18 μM and 6 downward 1/2-log dilutions. The third-passage stock of the SARS-CoV Toronto-2 strain was diluted to provide 100 TCID50 (tissue culture infectious dose) in the assay. For the inhibition and toxicity assays, the total volume of the solution in each well was 100 μL, with one-to-one drug dilution and virus or control suspension, respectively. Plates were incubated with 5% CO₂ at 37° C. for 72 hours after cells were exposed to a test compound and the virus. After incubation, cell viability was measured by a cytoprotection assay, which measures the ability of a test compound to prevent virus replication and subsequent cell death (Cytopathic Effect—CPE) by detecting the presence of viable cells. CellTiter-Glo Luminescent Cell Viability Assay by Promega (Madison, Wis.) was used for the detection of the number of viable cells in culture based on quantization of the ATP present, which signals the presence of metabolically active cells. The inhibition and toxicity assays were performed in triplicate and duplicate, respectively. For each plate, media controls were performed in replicates of eight; virus and cell controls were performed in replicates of five.

Models of SARS-CoV CCP. The energy minimizations of the hydrogen atoms of the two crystal structures were performed by using the SANDER module of the AMBER 5.0 program (Pearlman, D. A. et al., AMBER, a package of computer programs for applying molecular mechanics, normal mode analysis, molecular dynamics and free energy calculations to simulate the structural and energetic properties of molecules. Comput. Phys. Commun. 91, 1-41 (1995)), with maxcyc=10000, drms=0.01, ibelly=1, and ntmin=1. The minimizations were converged at 379 and 429 steps for the unbound (PDB code: 1UK2) and bound (PDB code: 1UK4) crystal structures, respectively.

Multiple Molecular Dynamics Simulations. All 4.0-ns molecular dynamics simulations of SARS-CoV CCP in complex with ATVRLQ^{p1}A^{p1′} were performed on 800 dedicated Intel Xeon P4 processors (2.2/2.4 GHz with hyperthreading) according to a published protocol (Pang, Y.-P., Three-dimensional model of a substrate-bound SARS chymotrypsin-like cysteine proteinase predicted by multiple molecular dynamics simulations: catalytic efficiency regulated by substrate binding. Proteins. 57, 747-757 (2004)) using the SANDER module of the AMBER 7.0 program with the Cornell et al. force field (parm96.dat) (Pearlman, D. A. et al., AMBER, a package of computer programs for applying molecular mechanics, normal mode analysis, molecular dynamics and free energy calculations to simulate the structural and energetic properties of molecules. Comput. Phys. Commun. 91, 1-41 (1995)). The topology and coordinate files used in the MMDSs were generated by the LINK, EDIT, and PARM modules of the AMBER 5.0 program (Pearlman, D. A. et al., AMBER, a package of computer programs for applying molecular mechanics, normal mode analysis, molecular dynamics and free energy calculations to simulate the structural and energetic properties of molecules. Comput. Phys. Commun. 91, 1-41 (1995)). All simulations used (1) a dielectric constant of 1.0, (2) the Berendsen coupling algorithm (Berendsen, H. J. C., Postma, J. P. M., van Gunsteren, W. F., Di Nola, A. & Haak, J. R., Molecular dynamics with coupling to an external bath. J. Chem. Phys. 81, 3684-3690 (1984)), (3) a periodic boundary condition at a constant temperature of 300 K and a constant pressure of 1 atm with isotropic molecule-based scaling, (4) the Particle Mesh Ewald method to calculate long-range electrostatic interactions (Darden, T. A., York, D. M. & Pedersen, L. G., Particle Mesh Ewald: An N log(N) method for Ewald sums in large systems. J. Chem. Phys. 98, 10089-10092 (1993)), (5) iwrap=1, (6) a time step of 1.0 fs, (7) the SHAKE-bond-length constraints applied to all the bonds involving the H atom, and (8) default values of all other inputs of the SANDER module. The reported ATVRLQ^{p1}A^{p1′}-bound CCP (Du, Q., Wang, S., Wei, D., Sirois, S. & Chou, K., Molecular modeling and chemical modification for finding peptide inhibitor against severe acute respiratory syndrome coronavirus main proteinase. Anal. Biochem. 337, 262-70 (2005)) was solvated with 8,713 TIP3P water molecules (Jorgensen, W. L., Chandreskhar, J., Madura, J. D., Impey, R. W. & Klein, M. L., Comparison of simple potential functions for simulating liquid water. J. Chem. Phys. 79, 926-935 (1982)) (EDIT input: NCUBE=20, QH=0.4170, DISO=2.20, DISH=2.00, CUTX=7.8, CUTY=8.0, and CUTZ=8.0). The solvated CCP complex had a total of 31,521 atoms and was first energy-minimized for 100 steps to remove close van der Waals contacts in the system, then slowly heated to 300 K (10 K/ps) and equilibrated for 1.5 ns. All energy minimizations used the default method of AMBER 7.0 (10 cycles of the steepest descent method followed by the conjugate gradient method).

Simulation Data Analysis. The CARNAL module of the AMBER 5.0 program (Pearlman, D. A. et al., AMBER, a package of computer programs for applying molecular mechanics, normal mode analysis, molecular dynamics and free energy calculations to simulate the structural and energetic properties of molecules. Comput. Phys. Commun. 91, 1-41 (1995)) was used for calculating the mass-weighted root mean square deviations and for obtaining the time-average structure. All graphics were generated with PyMOL (DeLano Scientific LLC) and Adobe Photoshop CS (Adobe System Incorporated).

Pharmacophore Analysis. Three-dimensional (3D) structures of CS11 and Tamiflu™ were generated by quantum chemistry calculations at the HF/6-31G* level using Gaussian 98 Program (Gaussian, Inc, Pittsburgh, Pa.). Overlay of the 3D structure of CS11 to that of Tamiflu™ was performed by using PyMOL (DeLano Scientific LLC).
Cell-Protection Data Analysis. Inhibition data were analyzed by using the Activity Base software from IDBS (Guildford, UK). Percentage of SARS-CoV inhibition by a test compound was calculated according to the following equation (Eq. 1):

$$\frac{( tcpd - mmc ) - ( mvc - mmc )}{( mcc - mmc ) - ( mvc - mmc )} * 100 \qquad ( 1 )$$

wherein tcpd represents the average measure of cells infected with virus and treated with a test compound, mmc represents the median measure of media control, mvc represents the median measure of virus infected cells, and mcc represents the median measure of cell control. Compounds were tested at 7 doses. Virus inhibition and cell viability were calculated for each dose. Percent cell viability (toxicity) was calculated according to the following equation (Eq. 2):

$$\frac{tcpd - mmc}{mcc - mmc} * 100 \qquad ( 2 )$$

wherein tcpd represents the average measure of cells infected with virus and treated with a test compound, mmc represents the median measure of media control and mcc represents the median measure of cell control. EC₅₀ (a drug concentration that produces 50% of inhibition) and CC₅₀ (a drug concentration that produces 50% of toxicity) were determined using the cumulative dose set by XLfit (version 4.1, formula 205) from IDBS (Guildford, UK).
Assay Quality Control.
A Z′ value was estimated for each plate using cells only as a positive control and cells infected with virus as a negative control. Z′ was calculated using the following equation (Eq. 3):

$$1 - \frac{3 \cdot SDp - 3 \cdot SDn}{\vert P - N \vert} \qquad ( 3 )$$

wherein P is the measure of untreated viable cells, N is the measure of viable cells infected with the virus, SDp is the standard deviation of the measure of untreated viable cells, and SDn is the standard deviation of the measure of cells infected with the virus. All reported data were obtained from experiments under the following conditions:
1) Calpain gave expected EC₅₀ values between 0.04 and 0.24 μM.
2) The median cell control values were more than twice the median virus control values.
3) Z′ was greater than 0.5.
   Detailed Information on Virtual Screening and Cell-Protection Assay. Chemical structures and protonation states of the compounds chosen for cell-based assays are shown in FIG. 1 (compounds identified using the 4.0-ns model). Intermolecular interaction energies and experimentally determined inhibition activities and cytotoxicities of these compounds are shown in Table 1.
   NMR Spectrum of CS11. Proton NMR spectrum was acquired on a Varian Mercury 400 (400 MHz) spectrometer. Chemical shifts are reported in ppm from the solvent resonance as the internal standard. Data are reported as follows: chemical shift, multiplicity (s=single, d=doublet, t=triplet, q=quartet, br=broad, m=multiplet), coupling constants (Hz), integration, and assignment. ¹H NMR (400 MHz, DMSO-d₆) d 13.39 (s, 1H, CO₂H), 9.16 (s, 1H, N═CH), 8.01 (d, J=8.4 Hz, 2H), 7.57 (m, 2H), 7.52 (d, J=8.4 Hz, 2H), 6.79 (d, J=8.8 Hz, 1H), 2.89 (m, 2H), 2.73 (m, 2H), and 1.83 (m, 4H).
   Results

To identify new inhibitor leads of CCP using genomic information instead of crystal structures of CCP with a flexible loop in the active site determined with low real-space correlation coefficients, a three-dimensional model in complex with a substrate fragment (ATVRLQ^{p1}A^{p1′}) was predicted by 200 molecular dynamics simulations (4.0 ns for each simulation with a 1.0-fs time step and different initial velocities) performed on terascale computers to predict different conformations of the flexible loop (residues 45-48) according to a published simulation protocol. An average structure of these simulations that represents CCP in the bound state was deposited to Protein Data Bank (PDB code: 2AJ5) and used as a drug target in virtual screening for small-molecule inhibitors, using a docking program, EUDOC.

Screening of 361,413 relatively rigid, small molecules against the 4.0-ns model of CCP identified 3,958 compounds with total and van der Waals interaction energies lower than −40 and −25 kcal/mol, respectively. The use of such energy cut-offs was based on the observations that all experimentally confirmed micromolar inhibitors identified by EUDOC had total and van der Waals interaction energies lower than the cutoffs. (Perola, E. et al., Successful virtual screening of a chemical database for farnesyltransferase inhibitor leads. J. Med. Chem. 43, 401-408 (2000); Pang, Y. P. et al., Discovery of a new inhibitor lead of adenovirus proteinase: steps toward selective, irreversible inhibitors of cysteine proteinases. FEBS Lett. 502, 93-97 (2001)). Twelve of these compounds were selected for experimental evaluation, after triaging compounds not commercially available and compounds with a large number of chiral centres, poor solubility, or poor cell permeability.

Of the twelve compounds tested in cell-based inhibition assays using African green monkey kidney (Vero E6) cells, one compound, CS11 (FIG. 1), inhibited the human SARS-CoV Toronto-2 strain with an EC₅₀ of 23 μM. Cell viability assays showed that this inhibitor was not toxic to normal cells at 23 μM. Four additional compounds (CS 08, 09, 10, and 12; see FIG. 1) showed 13-17% inhibition at a drug concentration of 32 μM. See Table 1, below.

TABLE 1
Computationally and Experimentally Determined Properties of
Compounds Identified Using a 4-ns Model

| | EUDOC | | | | |
|---|---|---|---|---|---|
| | calculated | | Average | | |
| | interaction | EUDOC | Inhibition | | |
| Inhibitor | energy | calculated VDW | (%) at | EC₅₀ | CC₅₀ |
| name | (kcal/mol) | energy (kcal/mol) | 32 μM | (μM) | (μM) |
| Calpain | NA | NA | 100 (5.7 μM) | 0.2 | 49 |
| CS01 | −71 | −33 | 0 | >100 | >100 |
| CS02 | −68 | −30 | 0 | >100 | >100 |
| CS03 | −64 | −39 | 0 | >100 | >100 |
| CS04 | −59 | −38 | 4 | >100 | >100 |
| CS05 | −59 | −38 | 0 | >100 | 10 |
| CS06 | −57 | −30 | 0 | >100 | 95 |
| CS07 | −54 | −38 | 1 | >100 | >100 |
| CS08 | −54 | −41 | 17 | >100 | >100 |
| CS09 | −53 | −32 | 13 | >100 | 85 |
| CS10 | −53 | −36 | 14 | >100 | 67 |
| CS11 | −48 | −34 | 66 | 23 | 76 |
| CS12 | −46 | −28 | 13 | >100 | >100 |

The result of the cell-based assay for CS11 agrees with the EUDOC-generated CS11-bound CCP complex. In the complex model, the cyclohexenyl and phenyl rings of CS11 occupy two hydrophobic regions of the active site, with the methylene and phenyl groups of CS11 mimicking the side chains of Leu^{P2} and Val^{P4} bound in a reported CCP complex; the carboxylate and hydroxyl groups of CS11 have hydrogen bonds with the amide proton of Gln192 and the carboxylate oxygen of Glu166. This model suggests that the potency of CS11 could be improved by minor structural modifications. For example, a replacement of the 4-aminobenzoic acid moiety of CS11 by a 4-amino-3-methylbenzoic acid could enhance the potency of CS11 due to the introduced methyl group better mimicking the side chain of Val^{P4}.

Superimposition of the three-dimensional structure of CS11 generated by the quantum chemical calculations onto that of Tamiflu™ shows that the hydrophobic groups and hydrogen-bond acceptors of the two molecules overlay well. Such structural properties indicate that the two molecules have similar pharmocophores, and suggest that CS11 and its analogs of Formulas I(a), I(b), II(a) and II(b) may be used for treating Avian Influenza infections including the H5N1 viral infections.

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising a compound according to Formula I(a): 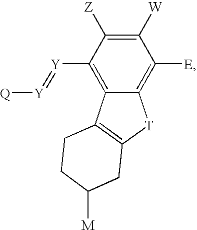
or a pharmaceutically acceptable salt or derivative thereof,
wherein Q is selected from: 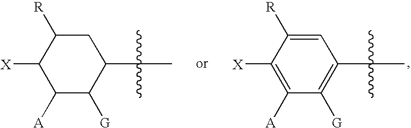
Y is N or CH;
X=H, CO₂H, NO₂, CH₂OH, COCH₃, CONH₂, or CONHCH₃;
Z=H, OH, F, NH₂, or aminoalkyl having from 1 to 5 carbon atoms;
A=H, or alkyl having from 1 to 5 carbon atoms;
G=H, or alkyl having from 1 to 5 carbon atoms;
R=H, OH, F, O(CH₂)ₘN(CH₃)₂, O(CH₂)ₘN(CH₃)H, O(CH₂)ₘNH₂, wherein m=1 to 5;
T is O, S, NH, or CO, or T is not present, in which case the adjacent cyclohexenyl ring is a cyclohexyl ring;
M=H or alkyl having from 1 to 3 carbon atoms, provided that if M=H, W=H, E=H, and T=O, then Z is not OH;
W=H, (CH₂)ₘNH₂, or (CH₂)ₘCONH₂, wherein m=1 to 5; and
E=H, alkyl having from 1 to 3 carbon atoms, CH₂CONH₂, or CH₂CONHR′, wherein R′ is alkyl having from 1 to 4 carbon atoms; or W and E together form a cyclohexyl ring which is fused to the adjacent phenyl ring, wherein said cyclohexyl ring is optionally substituted with X, or
a compound according to Formula I(b): 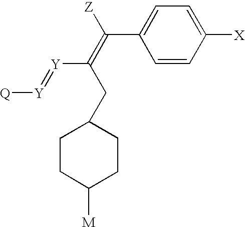
or a pharmaceutically acceptable salt or derivative thereof,
wherein Q is selected from: 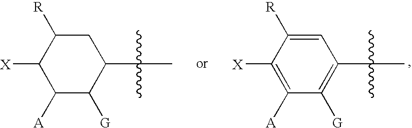
Y is N or CH;
X=H, CO₂H, NO₂, CH₂OH, COCH₃, CONH2, or CONHCH₃;
Z=H, OH, F, NH₂, or aminoalkyl having from 1 to 5 carbon atoms;
A=H, or alkyl having from 1 to 5 carbon atoms;
G=H, or alkyl having from 1 to 5 carbon atoms;
R=H, OH, F, O(CH₂)ₘN(CH₃)₂, O(CH₂)ₘN(CH₃)H, O(CH₂)ₘNH₂, wherein m=1 to 5; and
M=H or alkyl having from 1 to 3 carbon atoms.

2. The composition according to claim 1, wherein, with respect to Formula I(a), one Y is N and one Y is CH.

3. The composition according to claim 1, wherein, with respect to Formula I(a), T is O or S.

4. The composition according to claim 1, wherein, with respect to Formula I(a), T is not present and the cyclohexenyl group is a cyclohexyl group.

5. The composition according to claim 1, wherein, with respect to Formula I(b), one Y is CH and one Y is N.

6. The composition according to claim 1, wherein, with respect to Formula I(b), X is CO₂H.

7. The composition according to claim 1, wherein, with respect to Formula I(b), M is methyl.

8. The composition according to claim 1, wherein, with respect to Formula I(b), G is methyl.

9. The composition according to claim 1, having any of the structures of the compounds as set forth in FIG. 3.

10. A composition comprising a compound having the structure of CS11 as set forth in FIG. 1, or a pharmaceutically acceptable salt or derivative thereof, and an antiviral drug.

11. A composition comprising a compound according to Formula II(a): 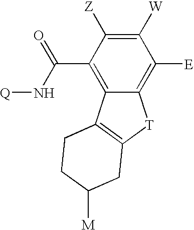
or a pharmaceutically acceptable salt or derivative thereof,
wherein Q is selected from: 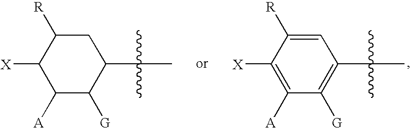
X=H, CO₂H, NO₂, CH₂OH, COCH₃, CONH2, or CONHCH₃;
Z=H, OH, F, NH₂, or aminoalkyl having from 1 to 5 carbon atoms;
A=H, or alkyl having from 1 to 5 carbon atoms;
G=H, or alkyl having from 1 to 5 carbon atoms;
R=H, OH, F, O(CH₂)ₘN(CH₃)₂, O(CH₂)ₘN(CH₃)H, O(CH₂)ₘNH₂, wherein m=1 to 5;
T is O, S, NH, or CO, or T is not present, in which case the adjacent cyclohexenyl ring is a cyclohexyl ring;
M=H or alkyl having from 1 to 3 carbon atoms;
W=H, (CH₂)ₘNH₂, or (CH₂)ₘCONH₂, wherein m=1 to 5; and
E=H, alkyl having from 1 to 3 carbon atoms, CH₂CONH₂, or CH₂CONHR′, wherein R′ is alkyl having from 1 to 4 carbon atoms; or W and E together form a cyclohexyl or phenyl ring which is fused to the adjacent phenyl ring, wherein said cyclohexyl or phenyl ring is optionally substituted with X; or
a compound according to Formula II(b): 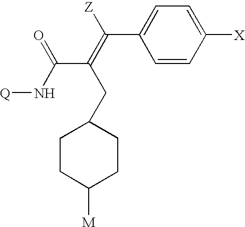
wherein Q is selected from: 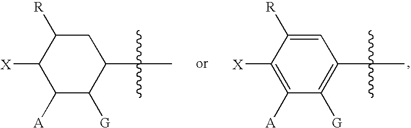
wherein X=H, CO₂H, NO₂, CH₂OH, COCH₃, CONH₂, or CONHCH₃;
Z=H, OH, F, NH₂, or aminoalkyl having from 1 to 5 carbon atoms;
A=H, or alkyl having from 1 to 5 carbon atoms;
G=H, or alkyl having from 1 to 5 carbon atoms;
R=H, OH, F, O(CH₂)ₘN(CH₃)₂, O(CH₂)ₘN(CH₃)H, O(CH₂)ₘNH₂, wherein m=1 to 5; and
M=H or alkyl having from 1 to 3 carbon atoms.

12. The composition of claim 11, wherein, with respect to Formula II(a), T is O or S.

13. The composition of claim 11, wherein, with respect to Formula II(a), X is CO₂H.

14. The composition of claim 11, wherein, with respect to Formula II(a), W and E form a cyclohexyl ring substituted with X.

15. The composition of claim 11, wherein, with respect to Formula II(a), T is not present and the cyclohexenyl group is a cyclohexyl group.

16. The composition of claim 11, wherein, with respect to Formula II(b), X is CO₂H.

17. The composition of claim 11, wherein, with respect to Formula II(b), Z is F.

18. The composition of claim 11, having any of the structures of the compounds set forth in FIG. 4.

19. A composition comprising a compound according to Formula III: 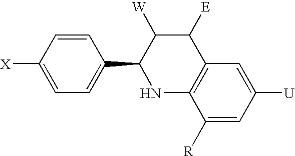
or a pharmaceutically acceptable salt or derivative thereof, wherein:
W and E together form a 5- or 6-membered cycloalkyl ring that is saturated or unsaturated and that is fused in any stereochemistry relative to the adjacent heterocyclyl ring;
X=H, CO₂H, NO₂, CH₂OH, COCH₃, CONH2, or CONHCH₃;
R=H, OH, F, O(CH₂)ₘN(CH₃)₂, O(CH₂)ₘN(CH₃)H, O(CH₂)ₘNH₂, wherein m=1 to 5; and
U=H, F, NH₂, NHR′, NHC(═O)R′, or N(R′)C(═O)R′, wherein R′ is alkyl having from 1 to 4 carbon atoms, provided that if U is NHC(═O)R′, where R′ is methyl, and R is H, then X is not NO₂.

20. The composition according to claim 19, wherein W and E form a 5-membered cycloalkyl ring that is unsaturated with one double bond.

21. The composition according to claim 19, wherein W and E form a 6-membered cycloalkyl ring that is unsaturated with one double bond.

22. The composition according to claim 19, wherein Formula III has the structure:

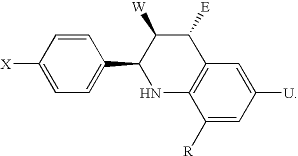

23. The composition according to claim 19, wherein X is NO₂.

24. The composition of claim 19, wherein U is F.

25. The composition of claim 19, wherein U is NHC(═O)C₂H₅.

26. A composition comprising a compound having the structure of CS08 or CS09 as set forth in FIG. 1, or a pharmaceutically acceptable salt or derivative thereof, and an antiviral drug.

27. A composition comprising a compound according to Formula IV: 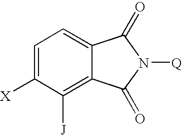
or a pharmaceutically acceptable salt or derivative thereof, wherein Q is: 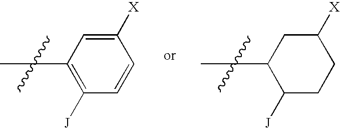
wherein X=H, CO₂H, CH₂OH, COCH₃, CONH2, or CONHCH₃; and
wherein J=H or alkyl having from 1 to 5 C atoms.

28. The composition of claim 27, wherein J is methyl.

29. The composition of claim 19 or claim 27, having the structure of any of the compounds as set forth in FIG. 5.

30. A composition comprising a compound having the structure of CS12 as set forth in FIG. 1 or a pharmaceutically acceptable salt or derivative thereof, and an antiviral drug.

31. A method of treating, preventing, or ameliorating one or more symptoms associated with a CoV infection comprising administering a composition according to claim 1 or claim 10 to a mammal.

32. A method of treating, preventing, or ameliorating one or more symptoms associated with a CoV infection comprising administering a composition according to claim 11 to a mammal.

33. A method of treating, preventing, or ameliorating one or more symptoms associated with CoV infection comprising administering a composition according to claim 19 or claim 26 to a mammal.

34. A method of treating, preventing, or ameliorating one or more symptoms associated with CoV infection comprising administering a composition according to claim 27 or claim 30 to a mammal.

35. The method of claim 31 wherein said CoV is human SARS CoV.

36. The method of claim 31 wherein said mammal is a human.

37. A method of treating, preventing, or ameliorating one or more symptoms associated with Avian Influenza, comprising administering a composition according to claim 1, 10, 11, 19, 26, 27, or 30, or a composition comprising a compound having the structure of CS08, CS09, CS11, or CS12, to a mammal or bird.

38. A method for inhibiting a chymotrypsin-like cysteine protease (CCP) activity comprising:
contacting a chymotrypsin-like cystein protease with a composition according to claim 1, 10, 11, 19, 26, 27, or 30, or with a composition comprising a compound having the structure of CS08, CS09, CS11, or CS12.

39. The method of claim 38, wherein said CCP is from human SARS CoV.

40. A kit comprising a composition according to claim 1, 10, 11, 19, 26, 27, or 30.

41. The kit of claim 40, wherein said composition is in the form of an injectable composition.

42. A composition according to claim 1, 10, 11, 19, 26, 27, or 30, or a composition including a compound having the structure of CS08, CS09, CS11, or CS12, for use in the treatment, prevention, or amelioration of CoV or Avian Influenza infection.

43. The composition of claim 42, wherein said CoV is human SARS CoV.

44. Use of a composition according to claim 1, 10, 11, 19, 26, 27, or 30, or a composition including a compound having the structure of CS08, CS09, CS11, or CS12, in the preparation of a medicament for the treatment, prevention, or amelioration of CoV or Avian Influenza infection.

45. The use of claim 44, wherein said CoV is human SARS CoV.

46. An article of manufacture comprising a composition according to claim 1, 10, 11, 19, 26, 27, or 30 disposed within a pill, a tablet, a capsule, or a syringe.
